(19) United States
(12) Patent Application Publication
(10) Pub. No.: US 2007/0249710 A1
(43) Pub. Date: Oct. 25, 2007

(54) Biaromatic compounds and cosmetic/pharmaceutical applications thereof

(75) Inventors: Jean-Claude Caron, Roquefort Les Pins (FR); Anne-Pascale Luzy, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/783,662

(22) Filed: Apr. 11, 2007

(30) Foreign Application Priority Data
Oct. 12, 2004 (FR) 0410749
Oct. 10, 2005 (FR) PCT/FR05/02487
Apr. 20, 2006 (WO) WO 2006/040462

(51) Int. Cl. A61K 31/24; C07C 251/56

(52) U.S. Cl. 514/534; 560/35

(57) ABSTRACT

Novel biaromatic compounds having the structural formula (I): 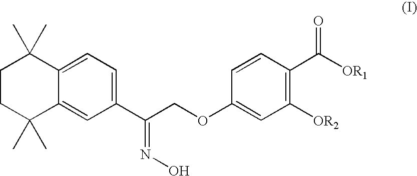 have a selective agonist activity with respect to the RAR-gamma receptor and are useful for combating skin aging and for treating, e.g., cell differentiation or proliferation disorders, pathologies related to keratinization disorders, acne and psoriasis.

### CROSS-REFERENCE TO PRIORITY/PCT APPLICATIONS

This application claims priority under 35 U.S.C. § 119 of FR 04/10749, filed Oct. 12, 2004, and is a continuation of PCT/FR 2005/002487, filed Oct. 10, 2005 and designating the United States (published in the French language on Apr. 20, 2006 as WO 2006/040462 A1; the title and abstract were also published in English), each hereby expressly incorporated by reference in its entirety and each assigned to the assignee hereof.

### BACKGROUND OF THE INVENTION

1. Technical Field of the Invention

The present invention relates to novel biaromatic compounds derived from a salicylic structural unit, to processes for preparing same and to their applications in human and veterinary medicine and in cosmetics.

2. Description of Background and/or Related and/or Prior Art:

A family of biaromatic compounds derived from a salicylic structural unit is described in EP-0,514,264. These compounds are described as having an application in the topical and systemic treatment of dermatological conditions related to a keratinization disorder and of opthalmological conditions in particular.

The activity of these compounds has in particular been demonstrated by means of mouse embryonic teratocarcinoma F9 cell differentiation assays and keratinocyte differentiation assays in humans.

On the other hand, this '264 document makes no reference to any possible specific activity of the compounds with respect to the RAR receptor gamma subtype.

### SUMMARY OF THE INVENTION

Surprisingly, it has now been demonstrated that 2-hydroxy-4-[2-hydroxyimino-2-(5,5,8,8-tetramethyl-5,6,7,8-tetrahydronaphthalen-2-yl)ethoxy]benzoic acid exhibits an extremely advantageous agonist activity that is selective for the gamma subtype of the RAR receptor family.

There is consequently an increased advantage in providing compounds that are prodrugs of this acid, possibly themselves exhibiting a selective activity with respect to the RARγ receptor.

The present invention provides such compounds, which also have the advantage of having a very short hepatic half-life time, advantageously on the order of 3 minutes, and of being completely compatible with a topical application.

According to a first embodiment, the present invention features novel compounds of general formula (I) 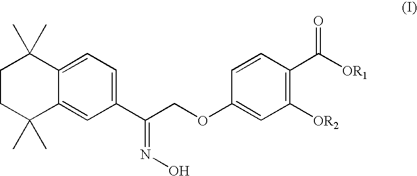
in which:

R₁ is an alkyl radical having from 2 to 20 carbon atoms, an alkenyl radical having from 2 to 20 carbon atoms, a mono- or polyhydroxyalkyl radical having from 1 to 6 carbon atoms, a sugar residue, or an amino acid residue; and

R₂ is a hydrogen atom or an alkyl radical having from 1 to 3 carbon atoms, and the salts of the compounds of formula (I), when R₁ is an amino acid residue, and the isomers of the compounds of formula (I).

The compounds of the invention of formula (I) defined above, which have a sufficiently acidic function or a sufficiently basic function or both, include the corresponding pharmaceutically acceptable salts of an organic or inorganic acid or of an organic or inorganic base.

This invention also relates to the tautomeric forms, to the enantiomers, diastereoisomers and epimers, and to the organic or inorganic salts of the compounds of general formula (I).

### BRIEF DESCRIPTION OF THE DRAWINGS

FIGS. 1 and 2 illustrate a variety of reaction schemes for the ultimate synthesis of the compounds of formula (I) according to the invention.

### DETAILED DESCRIPTION OF BEST MODE AND SPECIFIC/PREFERRED EMBODIMENTS OF THE INVENTION

As used above, and hereinafter, the following terms, unless otherwise indicated, should be understood to have the following meanings:

the term “alkyl radical” means an aliphatic hydrocarbon-based group which may be linear or branched, having from 2 to 20 carbon atoms in the chain. Preferred alkyl groups have from 2 to 12 carbon atoms in the chain, and in particular from 4 to 12 carbon atoms.

The term “branched alkyl” means that one or more lower alkyl group(s), such as methyl, ethyl or propyl, are attached to a linear alkyl chain.

The term “lower alkyl” is a radical having from 1 to 4 carbon atoms in the chain, which may be linear or branched.

Examples of alkyl radicals include, in particular, ethyl, isopropyl, n-propyl, tert-butyl, n-butyl, n-pentyl, n-hexyl, n-heptyl and n-octyl radicals.

The term “alkenyl” means an aliphatic hydrocarbon-based group which contains a carbon-carbon double bond and which may be linear or branched, having from 2 to 15 carbon atoms in the chain. Preferred alkenyl groups have 2 to 12 carbon atoms in the chain, and preferably from 2 to 4 carbon atoms in the chain.

The term “branched” means that one or more lower alkyl group(s), such as methyl, ethyl or propyl, is (are) attached to the linear alkenyl chain.

Examples of alkenyl radicals include, in particular, vinyl, allyl, 2-butenyl, n-butenyl, 3-methylbut-2-enyl, n-pentenyl, hexenyl, heptenyl, octenyl, cyclohexylbutenyl and decenyl radicals.

The term “monohydroxyalkyl radical” means an alkyl radical as defined herein, substituted with a hydroxyl group.

Examples of monohydroxyalkyl radicals include, in particular, hydroxymethyl, hydroxyethyl, hydroxypropyl, hydroxybutyl, hydroxypentyl and hydroxyhexyl radicals.

The term “polyhydroxyalkyl radical” means an alkyl radical as defined herein, preferably having from 2 to 6 carbon atoms, substituted with 2 to 5 hydroxyl groups. Examples of polyhydroxyalkyl radicals are, in particular, 2,3-dihydroxypropyl, 2,3,4-trihydroxybutyl and 2,3,4,5-tetrahydroxypentyl radicals.

For the purposes of the present invention, the term “residue of a sugar” means a residue that derives, for example, from glucose, galactose or mannose. Examples are, in particular, 6-glucosyl, 6-galactosyl and 6-mannosyl radicals.

The term “amino acid” is a group containing both an amino group and a carboxyl group, of general formula HOOC—CH (side chain) (NH₂).

The amino acids may be in the D, L or racemic configuration.

The amino acids comprise natural and synthetic amino acids.

The natural amino acids encompass the 20 amino acids that constitute proteins, such as serine, threonine and tyrosine.

The synthetic amino acids are known and encompass analogues of natural amino acids. In this respect, reference may in particular be made to Lehninger, A. L., Biochemistry, 2^{nd} ed., Worth Publishers, New York, 1975, 71-77.

The synthetic amino acids comprise amino acids in which the side chains have been replaced with synthetic derivatives.

In the context of the present invention, amino acids in which the side chain comprises one or more hydroxyl functions capable of forming, with a carboxylic acid function, an ester function are most particularly preferred. Examples of natural amino acids bearing a hydroxyl function are serine, threonine and tyrosine.

For the purposes of the present invention, the term “amino acid residue” means the residue of the amino acid after reaction of a hydroxyl group, borne by the side chain of the latter, with a carboxylic acid function to form an ester function of the type:

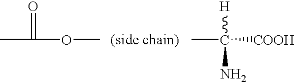

The expression “pharmaceutically acceptable salts” refers to the relatively non-toxic, inorganic or organic, acid addition salts, and the addition salts with a base, of the compounds of the present invention. These salts may be prepared in situ during the final isolation and the purification of the compounds. In particular, the acid addition salts can be prepared by separately reacting the purified compound in its purified form with an organic or inorganic acid, and isolating the salt thus formed.

Examples of acid addition salts include the following salts: hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamates, malonates, salicylates, propionates, methylenebis-b-hydroxynaphthoates, gentisic acid, isethionates, di-p-toluoyltartrates, methanesulfonates, ethanesulfonates, benzenesulfonates, p-toluenesulfonates, cyclohexyl sulfamates and quinates, lauryl sulfonate, and the like. (See, for example, S. M. Berge et al., “Pharmaceutical Salts” J. Pharm. Sci., 66: p. 1-19 (1977)). The acid addition salts can also be prepared by separately reacting the purified compound in its acid form, with an organic or inorganic base, and isolating the salt thus formed.

The acid addition salts include the amino and metal salts.

The suitable metal salts include the sodium, potassium, calcium, barium, zinc, magnesium and aluminum salts. The sodium and potassium salts are preferred.

The suitable inorganic addition salts with a base are prepared from metal bases which include sodium hydride, sodium hydroxide, potassium hydroxide, calcium hydroxide, aluminum hydroxide, lithium hydroxide, magnesium hydroxide and zinc hydroxide.

The suitable amino addition salts with a base are prepared from amines which have sufficient alkalinity to form a stable salt, and preferably comprise the amines which are often used in medicinal chemistry due to their low toxicity and their acceptability for medical applications: ammonia, ethylenediamine, N-methylglucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, for example lysine and arginine, and dicyclohexylamine, and the like.

The compounds of the present invention may contain asymmetrical centers. These asymmetrical centers may be independently in the R or S configuration. Certain compounds may also exhibit a geometric isomerism.

The present invention comprehends individual geometric isomers and stereoisomers and mixtures thereof, including racemic mixtures, of compounds of formula (I) above.

These types of isomers can be separated from their mixtures by the application or the adaptation of known processes, for example chromatography techniques or recrystallization techniques, or they are prepared separately from the appropriate isomers of their intermediates.

It is understood that, when reference is made to a given group, for example oxo/hydroxyl, this includes the tautomeric forms.

Preferably, R₁ is an alkyl radical, and in particular an alkyl radical having 2 to 6 carbon atoms.

According to one embodiment, R₂ is an alkyl group according to the definition indicated above and having from 1 to 3 carbon atoms, such as methyl, ethyl, n-propyl or isopropyl.

Preferably, R₂ is a hydrogen atom.

Among the compounds of formula (I) above, exemplary are the following compounds:
1) 2-hydroxy-4-[2-hydroxyimino-2-(5,5,8,8-tetramethyl-5,6,7,8-tetrahydronaphthalen-2-yl)ethoxy]benzoic acid ethyl ester;
2) 2-hydroxy-4-[2-hydroxyimino-2-(5,5,8,8-tetramethyl-5,6,7,8-tetrahydronaphthalen-2-yl)ethoxy]benzoic acid isopropyl ester;
3) 2-hydroxy-4-[2-hydroxyimino-2-(5,5,8,8-tetramethyl-5,6,7,8-tetrahydronaphthalen-2-yl)ethoxy]benzoic acid isobutyl ester;
4) 2-hydroxy-4-[2-hydroxyimino-2-(5,5,8,8-tetramethyl-5,6,7,8-tetrahydronaphthalen-2-yl)ethoxy]benzoic acid butyl ester;
5) 2-hydroxy-4-[2-hydroxyimino-2-(5,5,8,8-tetramethyl-5,6,7,8-tetrahydronaphthalen-2-yl)ethoxy]benzoic acid pentyl ester;
6) 2-hydroxy-4-[2-hydroxyimino-2-(5,5,8,8-tetramethyl-5,6,7,8-tetrahydronaphthalen-2-yl)ethoxy]benzoic acid hexyl ester;
7) 2-hydroxy-4-[2-hydroxyimino-2-(5,5,8,8-tetramethyl-5,6,7,8-tetrahydronaphthalen-2-yl)ethoxy]benzoic acid heptyl ester;
8) 2-hydroxy-4-[2-hydroxyimino-2-(5,5,8,8-tetramethyl-5,6,7,8-tetrahydronaphthalen-2-yl)ethoxy]benzoic acid hept-6-enyl ester;
9) 2-hydroxy-4-[2-hydroxyimino-2-(5,5,8,8-tetramethyl-5,6,7,8-tetrahydronaphthalen-2-yl)ethoxy]benzoic acid 2,3-dihydroxypropyl ester;
10) 2-hydroxy-4-[2-hydroxyimino-2-(5,5,8,8-tetramethyl-5,6,7,8-tetrahydronaphthalen-2-yl)ethoxy]benzoic acid 4-hydroxybutyl ester;
11) 2-hydroxy-4-[2-hydroxyimino-2-(5,5,8,8-tetramethyl-5,6,7,8-tetrahydronaphthalen-2-yl)ethoxy]benzoic acid 6-galactosyl ester;
12) 2-hydroxy-4-[2-hydroxyimino-2-(5,5,8,8-tetramethyl-5,6,7,8-tetrahydronaphthalen-2-yl)ethoxy]benzoic acid 6-glucosyl ester;
13) 2-hydroxy-4-[2-hydroxyimino-2-(5,5,8,8-tetramethyl-5,6,7,8-tetrahydronaphthalen-2-yl)ethoxy]benzoic acid 6-mannosyl ester;
14) 2-hydroxy-4-[2-hydroxyimino-2-(5,5,8,8-tetramethyl-5,6,7,8-tetrahydronaphthalen-2-yl)ethoxy]benzoic acid 2-amino-2-carboxyethyl ester;
15) 2-hydroxy-4-[2-hydroxyimino-2-(5,5,8,8-tetramethyl-5,6,7,8-tetrahydronaphthalen-2-yl)ethoxy]benzoic acid 4-(2-amino-2-carboxyethyl)phenyl ester; and
16) 2-hydroxy-4-[2-hydroxyimino-2-(5,5,8,8-tetramethyl-5,6,7,8-tetrahydronaphthalen-2-yl)ethoxy]benzoic acid 2-amino-2-carboxypropyl ester.

The compounds of general formula (I) can be prepared by application or adaptation of any method known per se from and/or within the scope of those skilled in the art, in particular those described by Larock in Comprehensive Organic Transformations, VCH Pub., 1989, or by application or adaptation of the processes described in the examples which follow, or else, more particularly, according to the method described in EP-0,514,264.

In the reactions described hereinafter, it may be necessary to protect the reactive functional groups, for example the hydroxyl, amino or carboxy groups, when they are desired in the final product, so as to prevent their unwanted participation in the reactions. Conventional protective groups may be used in accordance with standard practice; for examples see T. W. Green and P. G. M. Wuts in Protective Groups in Organic Chemistry, John Wiley and Sons, 1991; J. F. W. McOmie in Protective Groups in Organic Chemistry, Plenum Press, 1973.

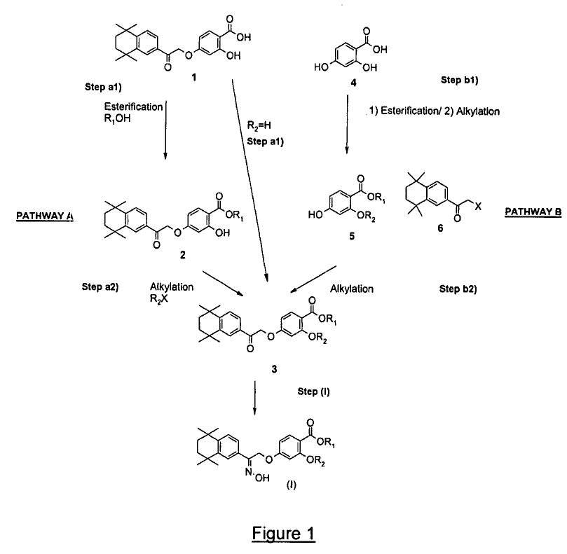

General synthetic pathways for preparing the compounds of formula (I) are represented in the scheme in FIG. 1.

The basic products or the reactants employed are commercially available and/or can be prepared by the application or adaptation of known processes, for example of the processes as described in the examples or their chemical equivalents.

The compounds of formula (I) can be prepared according to pathway A or pathway B as represented on the synthesis scheme of FIG. 1.

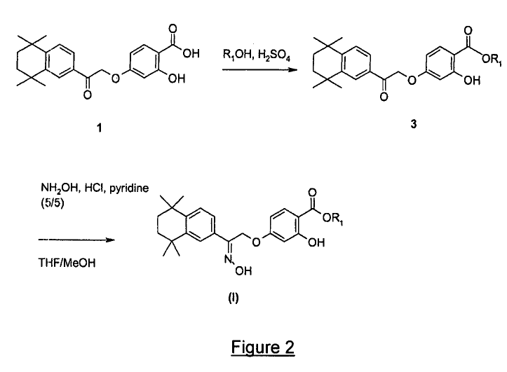

Unless otherwise indicated, all the substituents represented on the synthesis schemes of FIGS. 1 and 2 have the meanings indicated above.

According to another embodiment, the present invention therefore also features a process for preparing the compounds of formula (I) described above, comprising the following steps:

i) conversion of the ketone function of the compound of formula 3: 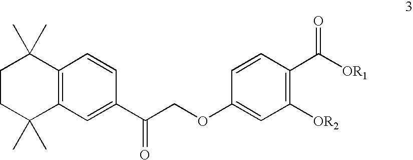
in which:

R₁ is an alkyl radical having from 2 to 20 carbon atoms, an alkenyl radical having from 2 to 20 carbon atoms, a mono- or polyhydroxyalkyl radical having from 1 to 6 carbon atoms, a sugar residue, or an amino acid residue; and

R₂ is a hydrogen atom or an alkyl radical having from 1 to 3 carbon atoms, to an oxime function, to produce the compound of formula (I): 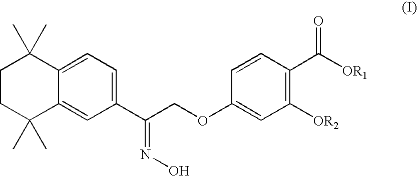
and, optionally,

ii) recovery of the compound of formula (I) obtained.

Step (i)

The conversion of the ketone function to an oxime can be carried out according to known methods.

By way of example, the oxime can be prepared by reaction of hydroxylamine, or of a salt thereof, such as a hydroxylamine hydrohalide, and in particular hydroxylamine hydrochloride, on the ketone.

Hydroxylamine derivatives such as, for example, H₂NOSO₃H and HON(SO₃Na)₂ can also be used. In this respect, reference is made to March, Jerry, Advanced Organic Chemistry, 3^{rd} Ed., John Wiley and Sons.

The ratio of hydroxylamine, or of its salts or of its derivatives, to the ketone compound 3 can vary, for example, within the range of from 1 to 20 molar equivalents, more preferably from 1 to 5 molar equivalents. The amount of hydroxylamine hydrochloride and of pyridine is, for example, respectively 1 mol per mole of compound 3.

The oxime may also be prepared by reaction of the compound 3 with a combination of hydroxylamine halohydride and of pyridine.

Preferably, these two reactants are present in molar equivalent proportions (1/1) respectively.

Step (i) can also be carried out in the presence of an appropriate base. There is no specific restriction in terms of the nature of the base in this reaction, and any base conventionally used in reactions of this type can be used here, provided that it has no adverse effect on the other parts of the molecule.

Examples of appropriate bases are in particular alkali metal hydroxides, such as potassium hydroxide or sodium hydroxide, and amines such as triethylamine or diisopropylethylamine.

There is no specific restriction in terms of the nature of the solvent to be used, provided that it has no adverse effect on the reaction or the reactants involved.

Examples of suitable solvents are in particular polar solvents, including, in particular, aliphatic alcohols such as methanol, ethanol or isopropanol; aliphatic ethers such as tetrahydrofuran (THF), diethyl ether, dibutyl ether or dioxane; and mixtures of these solvents.

The reaction may be carried out over a very broad range of temperatures, and it is not essential to the invention that the reaction be carried out at a specific temperature. The reaction temperature is, for example, from 15° C. and 150° C., and most commonly from 20° C. and 100° C.

The reaction may, for example, be carried out at the reflux of methanol and/or of THF.

Pathway A:

Step a1):

According to a first specific embodiment of the process of the invention, the compound of formula 3 is prepared according to a process comprising a reaction entailing esterification of the acid 1, according to a step a1), in the presence of an alcohol R₁OH, where R₁ has the definition indicated above, whereby a compound of formula 2 is obtained.

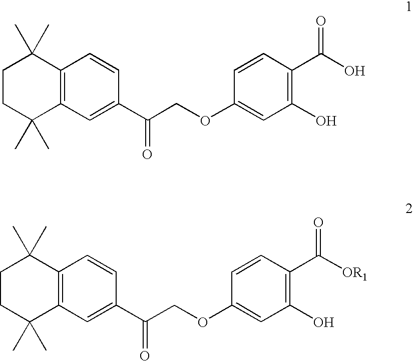

The esterification reaction according to step a1) can be catalyzed by an appropriate acid. Examples of an acid are, in particular, hydrochloric acid, hydrobromic acid, nitric acid or sulfuric acid, the latter being preferred.

When R₂═H, the compound of formula 3 is thus directly obtained, according to step a1), from the 2-hydroxy-4-[2-hydroxyimino-2-(5,5,8,8-tetramethyl-5,6,7,8-tetrahydronaphthalen-2-yl)ethoxy]benzoic acid 1. The latter can be prepared according to the method described in EP-0,514,264.

Step a2):

When R₂ is an alkyl radical, the process for preparing the compound of formula 3 also comprises a reaction entailing alkylation of the phenol function of the compound 2 in the presence of an alkyl halide of formula R₂X, in which R₂ is an alkyl radical having from 1 to 3 carbon atoms, and X is a halogen atom such as Cl or Br.

This alkylation reaction can be carried out according to conventional methods. In this respect, reference is in particular made to March, Jerry, Advanced Organic Chemistry, 3^{rd} Ed., John Wiley and Sons.

For example, the alkylation reaction can be carried out in the presence of an appropriate base.

Examples of an appropriate base are, in particular, alkali metal carbonates such as potassium carbonate, sodium carbonate or caesium carbonate; and alkali metal hydroxides such as sodium hydroxide or potassium hydroxide.

Pathway B:

Step b2):

According to a second embodiment, the compound 3 is prepared by means of a reaction entailing alkylation of a haloacetophenone 6, in particular bromoacetophenone, with a compound of formula 5.

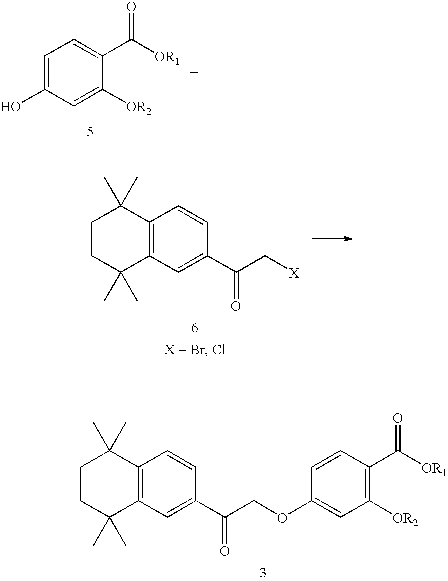

This alkylation reaction can be carried out according to known methods. In this respect, reference is in particular made to March, Jerry, Advanced Organic Chemistry, 3^{rd} Ed., John Wiley and Sons.

For example, the alkylation reaction can be carried out in the presence of an appropriate base.

Examples of an appropriate base are, in particular, alkali metal carbonates such as potassium carbonate, sodium carbonate or caesium carbonate, alkali metal hydroxides such as sodium hydroxide or potassium hydroxide, or potassium carbonate, the latter being particularly preferred.

Step b1):

The compound 5 can be prepared by means of a reaction entailing esterification of dihydrobenzoic acid with an alcohol R₁OH, followed by a reaction consisting of alkylation with an alkyl halide R₂X, according to conventional methods.

According to a particularly preferred embodiment, in the case where R₂═H, the compounds of formula (I) are prepared according to the following steps:

esterification of 2-hydroxy-4-[2-hydroxyimino-2-(5,5,8,8-tetramethyl-5,6,7,8-tetrahydronaphthalen-2-yl)ethoxy]benzoic acid of formula 1 in the presence of sulfuric acid and of an alcohol R₁OH; and

reaction of the ester obtained in step 1) with a mixture of hydroxylamine hydrochloride and of pyridine in a polar solvent.

This embodiment is in particular illustrated by the synthesis scheme of FIG. 2.

The compound (I) thus prepared and, where appropriate, the intermediates 2 and 5 can be recovered from the reaction mixture by conventional means. For example, the compounds can be recovered by distilling the solvent of the reaction mixture or, if necessary, after distillation of the solvent of the solution mixture, by pouring the rest into water, followed by extraction with a water-immiscible organic solvent, and distilling the solvent of the extract. In addition, the product may, if desired, be further purified by various techniques, such as recrystallization, reprecipitation or various chromatography techniques, in particular preparative thin layer chromatography or column chromatography.

The present invention also features administration of the compounds of formula (I) described above as medicaments.

According to another embodiment, this invention features pharmaceutical or cosmetic compositions, comprising, in a pharmaceutically or cosmetically acceptable carrier, at least one compound of formula (I) above.

The expression “pharmaceutically or cosmetically acceptable carrier” means a carrier suitable for use in contact with human and animal cells, without undue toxicity, irritation and allergic response and the like, and adjusted to a reasonable advantage/risk ratio.

The administration, whether regime or regimen, may be carried out topically, enterally or orally, parenterally or ocularly.

Among these routes of administration, topical administration is particularly preferred.

When administered topically, the pharmaceutical composition according to the invention is more particularly for the treatment of the skin and the mucous membranes, and may be in liquid, pasty or solid form, and more particularly in the form of salves, creams, milks, ointments, powders, impregnated pads, syndets, solutions, gels, sprays, foams, suspensions, sticks, shampoos or washing bases. It may also be in the form of suspensions of microspheres or nanospheres or of vesicles formed from lipid or polymer or of polymeric or gel patches for controlled release.

The compounds are administered topically at a concentration generally of from 0.001% and 3% by weight, relative to the total weight of the composition.

For a cosmetic application, the composition is preferably in the form of a cream, a milk, a lotion, a gel, microspheres or nanospheres or vesicles formed from lipid or polymer, a soap or a shampoo.

When administered enterally or orally, the composition may be in the form of tablets, gels, sugar-coated tablets, syrups, suspensions, solutions, powders, granules, emulsions, suspensions of microspheres or nanospheres or vesicles formed from lipid or polymer for controlled release. When administered parenterally, the composition may be in the form of solutions or suspensions for infusion or for injection.

The compounds according to the invention are generally administered at a daily dose of approximately 0.01 mg/kg to 30 mg/kg of body weight, taken as 1 to 3 doses.

The compounds according to the invention are advantageously prodrugs.

The term “prodrugs” signifies that the compounds are converted in vivo, to give the parent compound, 2-hydroxy-4-[2-hydroxyimino-2-(5,5,8,8-tetramethyl-5,6,7,8-tetrahydronaphthalen-2-yl)ethoxy]benzoic acid, by hydrolysis in the human or animal body (Prodrugs, Drugs and the Pharmaceutical Sciences, Kenneth B. Sloan, Vol. 53, p. 152).

The compounds of the invention can be formulated, alone or as a mixture, into pharmaceutical or cosmetic compositions in which said compound is an agent that exerts a selective agonist effect with respect to the RAR-gamma receptor.

The present invention also features a method of therapeutic or cosmetic treatment, comprising the administration of a pharmaceutical or cosmetic composition comprising such a compound, as an agent that exerts a selective agonist activity with respect to the RAR-gamma receptor.

The pharmaceutical composition may be more particularly for treating a pathology for the treatment of which a selective agonist activity with respect to the RAR-gamma receptor is desired.

The composition can also be for the treatment of a pathology related to cell differentiation or proliferation disorders, in particular in the field of dermatology.

More particularly, it can be for the treatment of a pathology related to a keratinization disorder.

The treatment of acne is thus envisaged, in particular common acne, comedone acne, polymorphic acne, nodulocystic acne, acne conglobata, senile acne, or secondary acne such as solar acne, acne medicamentosa or occupational acne.

The pharmaceutical composition comprising a compound of the invention is also useful for treating other dermatological conditions related to a keratinization disorder with an inflammatory and/or immunoallergic component, and in particular all forms of psoriasis, whether they are cutaneous, mucosal or ungueal.

The compounds of the invention are also useful in a cosmetic composition, for combating skin aging, whether it is, for example, photo-induced or chronological aging.

The pharmaceutical or cosmetic composition is also useful to reduce skin pigmentations and to treat actinic keratoses.

In all the applications envisaged, the compound of the invention can be combined with another therapeutic agent for the treatment of a pathology related to cell differentiation or proliferation disorders.

In order to further illustrate the present invention and the advantages thereof, the following specific examples are given, it being understood that same are intended only as illustrative and in nowise limitative. In said examples to follow, all parts and percentages are given by weight, unless otherwise indicated.

In the examples hereinafter, the samples were analyzed by ¹H NMR, ¹³C NMR and HPLC/MS.

### EXAMPLE 1

### Synthesis of ethyl 2-hydroxy-4-[2-hydroxyimino-2-(5,5,8,8-tetramethyl-5,6,7,8-tetrahydronaphthalen-2-yl)ethoxy]benzoate

### Preparation of ethyl 2-hydroxy-4-[2-oxo-2-(5,5,8,8-tetramethyl-5,6,7,8-tetrahydronaphthalen-2-yl)ethoxy]benzoate

5 g (13 mmol) of 2-hydroxy-4-[2-oxo-2-(5,5,8,8-tetramethyl-5,6,7,8-tetrahydronaphthalen-2-yl)ethoxy]benzoic acid are dissolved in 100 ml of absolute ethanol. 1 ml of concentrated sulfuric acid is added, and the reaction medium is stirred at reflux temperature for 24 hours. After hydrolysis and extraction with ethyl acetate, the organic phase is dried and concentrated, and the residue is then purified by chromatography (eluent: 95 heptane/5 EtOAc). A yellow oil is obtained (3.9 g, R=73%).

### Synthesis of ethyl 2-hydroxy-4-[2-hydroxyimino-2-(5,5,8,8-tetramethyl-5,6,7,8-tetrahydronaphthalen-2-yl)ethoxy]benzoate

3.9 g (9.5 mmol) of ethyl 2-hydroxy-4-[2-oxo-2-(5,5,8,8-tetramethyl-5,6,7,8-tetrahydronaphthalen-2-yl)ethoxy]benzoate are dissolved in 100 ml of THF and 100 ml of methanol. 3.9 ml (47.5 mmol) of pyridine followed by 3.3 g (47.5 mmol) of hydroxylamine hydrochloride are added, and the medium is stirred at reflux temperature for 1 h 30. The reaction medium is treated with a 1N hydrochloric acid solution and extracted with ethyl acetate. The residue obtained after drying and concentrating is purified by silica column chromatography (eluent: 95 heptane/5 EtOAc). Two products are obtained: the syn isomer (white solid, Mp 130° C., m=2.4 g, R=59%), ¹H NMR (CDCL₃): 1.29 (s, 12H); 1.39-1.42 (t, J=8 Hz, 3H); 1.69 (s, 4H); 4.40 (q, J=8 Hz, 2H); 5.29 (s, 2H); 6.48 (dd, J1=4 Hz, J2=8 Hz, 1H); 6.57 (s, 1H); 7.30-7.32 (m, 1H); 7.40-7.42 (m, 1M); 7.61 (s, 1H); 7.74-7.76 (d, J=8 Hz, 1H); 8.20 (s, 1H); 11.0 (s, 1H), and the anti isomer (white solid, Mp=155° C., m=0.5 g, R=12%) ¹H NMR (CDCl₃): 1.27 (s, 6H); 1.30 (s, 6H); 1.39-1.43 (t, J=8 Hz, 3H); 1.7 (s, 4H); 4.36-4.42 (q, J=8 Hz, 2H); 4.92 (s, 2H); 6.4-6.52 (m, 1H); 6.55-6.56 (s, 1H); 7.35-7.42 (m, 2H); 7.58 (s, 1H); 7.63 (s, 1H); 7.76-7.78 (d, J=8 Hz, 1H); 11 (s, 1H).

### EXAMPLE 2

### Synthesis of isopropyl 2-hydroxy-4-[2-hydroxyimino-2-(5,5,8,8-tetramethyl-5,6,7,8-tetrahydronaphthalen-2-yl)ethoxy]benzoate

### Preparation of isopropyl 2-hydroxy-4-[2-oxo-2-(5,5,8,8-tetramethyl-5,6,7,8-tetrahydronaphthalen-2-yl)ethoxy]benzoate

In a manner similar to Example 1a, by reacting 1.9 g (5 mmol) of 2-hydroxy-4-[2-oxo-2-(5,5,8,8-tetramethyl-5,6,7,8-tetrahydronaphthalen-2-yl)ethoxy]benzoic acid in 50 ml of isopropanol. A brown oil is obtained (m=660 mg, R=32%).

### Synthesis of isopropyl 2-hydroxy-4-[2-hydroxyimino-2-(5,5,8,8-tetramethyl-5,6,7,8-tetrahydronaphthalen-2-yl)ethoxy]benzoate

In a manner similar to Example 1b, by reacting 660 mg (1.5 mmol) of isopropyl 2-hydroxy-4-[2-oxo-2-(5,5,8,8-tetramethyl-5,6,7,8-tetrahydronaphthalen-2-yl)ethoxy]benzoate with 0.54 g (78 mmol) of hydroxylamine hydrochloride and 0.6 ml of pyridine. Two products are obtained: the syn isomer (white solid, Mp 130° C., m=600 mg, R=88%), ¹H NMR (CDCl₃): 1.28 (s, 12H); 1.37-1.38 (d, J=6.25, 6H); 2.19 (s, 4H); 5.29 (m, 3H); 6.49 (m, 1H); 6.57 (s, 1H); 7.29-7.31 (m, 1H); 7.40 (m, 1H); 7.61 (s, 1H); 7.72-7.75 (d, J=8 Hz, 1H); 8.45 (s, 1H); 11 (s, 1H) and the anti isomer (white solid, Mp=149° C., m=70 mg, R=5%) ¹H NMR (CDCl₃): 1.27 (s, 6H); 1.30 (s, 6H); 1.38-1.39 (d, J=6.24 Hz, 6H); 1.70 (s, 4H); 4.9 (s, 2H); 5.25-5.29 (m, 1H); 6.48-6.51 (m, 1H); 6.55 (s, 1H); 7.35-7.42 (m, 2H); 7.58 (s, 1H); 7.59-7.61 (m, 1H); 7.74-7.77 (d, J=8 Hz, 1H), 11 (s, 1H).

### EXAMPLE 3

### Synthesis of isobutyl 2-hydroxy-4-[2-hydroxyimino-2-(5,5,8,8-tetramethyl-5,6,7,8-tetrahydronaphthalen-2-yl)ethoxy]benzoate

### Preparation of isobutyl 2-hydroxy-4-[2-oxo-2-(5,5,8,8-tetramethyl-5,6,7,8-tetrahydronaphthalen-2-yl)ethoxy]benzoate

In a manner similar to Example 1a, by reacting 1.9 g (5 mmol) of 2-hydroxy-4-[2-oxo-2-(5,5,8,8-tetramethyl-5,6,7,8-tetrahydronaphthalen-2-yl)ethoxy]benzoic acid in 50 ml of isobutanol. A brown oil is obtained (m=1.7 g, R=79%).

### Synthesis of isobutyl 2-hydroxy-4-[2-hydroxyimino-2-(5,5,8,8-tetramethyl-5,6,7,8-tetrahydronaphthalen-2-yl)ethoxy]benzoate

In a manner similar to Example 1b, by reacting 1.7 g (3.9 mmol) of isobutyl 2-hydroxy-4-[2-oxo-2-(5,5,8,8-tetramethyl-5,6,7,8-tetrahydronaphthalen-2-yl)ethoxy]benzoate with 1.4 g (20 mmol) of hydroxylamine hydrochloride and 1.6 ml of pyridine. Two products are obtained: the syn isomer (white solid, Mp 96° C., m=1.2 g, R=70%), ¹H NMR (CDCl₃): 1.02-1.04 (d, J=8 Hz, 6H); 1.29 (s, 12H); 1.69 (s, 4H); 2.1-2.2 (m, 1H); 4.10-4.12 (d, J=8 Hz, 2H); 5.29 (s, 2H); 6.48-6.50 (dd, J=8 Hz, 1H); 6.58 (s, 1H); 7.30-7.32 (m, 1H); 7.40-7.42 (m, 1H); 7.62 (s, 1H); 7.75-7.77 (d, J=8 Hz, 1H); 8.21 (s, 1H); 11 (s, 1H), and the anti isomer (white solid, Mp=156° C., m=130 mg, R=7%) ¹H NMR (CDCl₃): 1.03-1.05 (d, J=6.7 Hz, 6H); 1.28 (s, 6H); 1.30 (s, 6H); 1.70 (s, 4H); 2.08-2.11 (m, 1H); 4.11-4.12 (d, J=6.7 Hz, 2H); 4.9 (s, 2H); 6.5-6.52 (m, 1H); 6.56 (s, 1H); 7.35-7.42 (m, 2H); 7.59 (s, 1H); 7.66 (s, 1H); 7.76-7.78 (d, J=8 Hz, 1H); 11 (s, 1H).

### EXAMPLE 4

### Transactivation Test

Principle of the Test:

The activation of the receptors by an agonist (activator) in HeLa cells results in the expression of a reporter gene, luciferase, which, in the presence of a substrate, generates light. It is therefore possible to measure the activation of the receptors by quantifying the luminescence produced after incubation of the cells in the presence of a reference antagonist. The activator products displace the antagonist from its site, thus allowing activation of the receptor. The measurement of the activity is carried out by quantifying the increase in the light produced. This measurement makes it possible to determine the activating activity of the compound for use in the invention.

In this study, a constant which is the affinity of the molecule for the receptor is determined. Since this value can fluctuate depending on the basal activity and the expression of the receptor, it is designated apparent Kd (KdApp).

To determine this constant, “cross curves” for the product to be tested (2-hydroxy-4-[2-hydroxyimino-2-(5,5,8,8-tetramethyl-5,6,7,8-tetrahydronaphthalen-2-yl)ethoxy]benzoic acid), against a reference antagonist, otherwise called reference ligand, 4-(5,5-dimethyl-8-p-tolyl-5,6-dihydronaphthalen-2-ylethynyl)benzoic acid, are produced. The product to be tested is used at 10 concentrations and the reference antagonist at 7 concentrations. In each well (of a 96-well plate), the cells are in contact with one concentration of the product to be tested and one concentration of the reference antagonist.

Measurements are also carried out for the total agonist control, otherwise called 100% control (4-[2-(5,5,8,8-tetramethyl-5,6,7,8-tetrahydronaphthalen-2-yl)propenyl]benzoic acid), and the inverse agonist control, otherwise called 0% control, 4-{(E)-3-[4-(4-tert-butylphenyl)-5,5,8,8-tetramethyl-5,6,7,8-tetrahydronaphthalen-2-yl]-3-oxopropenyl}benzoic acid.

These cross curves make it possible to determine the AC50 values (concentration at which 50% activation is observed) for the reference ligand at various concentrations of product to be tested. These AC50 values are used to calculate the Schild regression by plotting a straight line corresponding to the Schild equation (“quantitation in receptor pharmacology”, Terry P. Kenakin, Receptors and Channels, 2001, 7, 371-385).

In the case of an agonist, the AC50 value (concentration that gives 50% of the activity) is calculated by plotting the curve of the product at the concentration of the reference ligand that gives 80% activation. The percentage activation which corresponds to the maximum level of activity obtained is also measured.

Materials and Method:

The HeLa cell lines used are stable transfectants containing the plasmids ERE-βGlob-Luc-SV-Neo (reporter gene) and RAR (α, β, γ) ER-DBD-puro. These cells are seeded into 96-well plates at a rate of 10,000 cells per well in 100 μl of DMEM medium without phenol red, supplemented with 10% of delipidized calf serum. The plates are then incubated at 37° C., 7% CO₂ for 4 hours.

The various dilutions of the product to be tested, of the reference ligand (4-(5,5-dimethyl-8-p-tolyl-5,6-dihydronaphthalen-2-ylethynyl)benzoic acid), of the 100% control (100 nM 4-[2-(5,5,8,8-tetramethyl-5,6,7,8-tetrahydronaphthalen-2-yl)propenyl]benzoic acid) and of the 0% control (500 nM 4-{(E)-3-[4-(4-tert-butylphenyl)-5,5,8,8-tetramethyl-5,6,7,8-tetrahydronaphthalen-2-yl]-3-oxopropenyl}benzoic acid) are added at a rate of 5 μl per well. The plates are then incubated for 18 hours at 37° C., 7% CO₂.

The culture medium is removed by turning the plates upside down and 100 μl of a 1:1 PBS (phosphate buffer solution)/luciferin mixture are added to each well. After 5 minutes, the plates are read with a luminescence reader.

Results:

The values of the apparent Kd constants are reported in the table hereinafter.

| | RARalpha | | | RARbeta | | | RARgamma | | |
|---|---|---|---|---|---|---|---|---|---|
| | | | % | | | % | | | % |
| | Kdapp | AC50 | activation | Kdapp | AC50 | Activation | Kdapp | AC50 | activation |
| | (nM) | (nM) | (at 10 μM) | (nM) | (nM) | (at 5 μM) | (nM) | (nM) | (at 0.04 μM) |
| Acid | 4060 | 1700 | 100 | 1030 | 600 | 100 | 8 | 9 | 100 |
| compound | | | | | | | | | |
| of the | | | | | | | | | |
| invention | | | | | | | | | |

The results obtained with 2-hydroxy-4-[2-hydroxyimino-2-(5,5,8,8-tetramethyl-5,6,7,8-tetrahydronaphthalen-2-yl)ethoxy]benzoic acid clearly show that this compound has great specificity for the receptor subtype RARgamma in comparison with the other two subtypes RARalpha and RARbeta.

2-Hydroxy-4-[2-hydroxyimino-2-(5,5,8,8-tetramethyl-5,6,7,8-tetrahydronaphthalen-2-yl)ethoxy]benzoic acid is a selective agonist or activator of the RARgamma receptor.

### EXAMPLE 5

### Irritation Test

The irritant capacity of the ethyl ester and of the isobutyl ester of 2-hydroxy-4-[2-hydroxyimino-2-(5,5,8,8-tetramethyl-5,6,7,8-tetrahydronaphthalen-2-yl)ethoxy]benzoic acid (compounds of Examples 1 and 3, respectively) is evaluated by single topical application to the ear in BALB/c mice. The mice (ByJIco) are 8-week-old female mice. A batch of 5 mice is tested.

20 μl of the product to be tested, in acetone, is applied to the right ear at D1. The thickness of the ear is measured using the Oditest at times D4, D5, D6, D7, D8 and D11.

Neither of the two compounds tested induces any irritation at the tested doses of 0.003% to 0.01%.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A biaromatic compound having the structural formula (I): 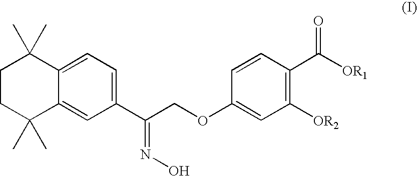
in which:
R₁ is an alkyl radical having from 2 to 20 carbon atoms, an alkenyl radical having from 2 to 20 carbon atoms, a mono- or polyhydroxyalkyl radical having from 1 to 6 carbon atoms, a sugar residue, or an amino acid residue; and
R₂ is a hydrogen atom or an alkyl radical having from 1 to 3 carbon atoms,
and the salts of the compounds of formula (I), when R₁ is an amino acid residue, and the isomers of the compounds of formula (I).

2. The biaromatic compound as defined by claim 1, wherein formula (I) R₁ is selected from the group consisting of ethyl, isopropyl, n-propyl, tert-butyl, n-butyl, n-pentyl, n-hexyl, n-heptyl and n-octyl radicals.

3. The biaromatic compound as defined by claim 1, wherein formula (I) R₁ is selected from the group consisting of vinyl, allyl, 2-butenyl, n-butenyl, 3-methylbut-2-enyl, n-pentenyl, hexenyl, heptenyl, octenyl, cyclohexylbutenyl and decenyl radicals.

4. The biaromatic compound as defined by claim 1, wherein formula (I) R₁ is selected from the group consisting of hydroxymethyl, hydroxyethyl, hydroxypropyl, hydroxybutyl, hydroxypentyl and hydroxyhexyl radicals.

5. The biaromatic compound as defined by claim 1, wherein formula (I) R₁ is selected from the group consisting of 2,3-dihydroxypropyl, 2,3,4-trihydroxybutyl and 2,3,4,5-tetrahydroxypentyl radicals.

6. The biaromatic compound as defined by claim 1, wherein formula (I) R₁ is selected from the group consisting of 6-glucosyl, 6-galactosyl and 6-mannosyl radicals.

7. The biaromatic compound as defined by claim 1, wherein formula (I) R₁ is selected from the group consisting of serine, tyrosine and threonine radicals.

8. The biaromatic compound as defined by claim 1, wherein formula (I) R₂ is a hydrogen atom.

9. The biaromatic compound as defined by claim 1, selected from the group consisting of the following compounds:
1) 2-hydroxy-4-[2-hydroxyimino-2-(5,5,8,8-tetramethyl-5,6,7,8-tetrahydronaphthalen-2-yl)ethoxy]benzoic acid ethyl ester;
2) 2-hydroxy-4-[2-hydroxyimino-2-(5,5,8,8-tetramethyl-5,6,7,8-tetrahydronaphthalen-2-yl)ethoxy]benzoic acid isopropyl ester;
3) 2-hydroxy-4-[2-hydroxyimino-2-(5,5,8,8-tetramethyl-5,6,7,8-tetrahydronaphthalen-2-yl)ethoxy]benzoic acid isobutyl ester;
4) 2-hydroxy-4-[2-hydroxyimino-2-(5,5,8,8-tetramethyl-5,6,7,8-tetrahydronaphthalen-2-yl)ethoxy]benzoic acid butyl ester;
5) 2-hydroxy-4-[2-hydroxyimino-2-(5,5,8,8-tetramethyl-5,6,7,8-tetrahydronaphthalen-2-yl)ethoxy]benzoic acid pentyl ester;
6) 2-hydroxy-4-[2-hydroxyimino-2-(5,5,8,8-tetramethyl-5,6,7,8-tetrahydronaphthalen-2-yl)ethoxy]benzoic acid hexyl ester;
7) 2-hydroxy-4-[2-hydroxyimino-2-(5,5,8,8-tetramethyl-5,6,7,8-tetrahydronaphthalen-2-yl)ethoxy]benzoic acid heptyl ester;
8) 2-hydroxy-4-[2-hydroxyimino-2-(5,5,8,8-tetramethyl-5,6,7,8-tetrahydronaphthalen-2-yl)ethoxy]benzoic acid hept-6-enyl ester;
9) 2-hydroxy-4-[2-hydroxyimino-2-(5,5,8,8-tetramethyl-5,6,7,8-tetrahydronaphthalen-2-yl)ethoxy]benzoic acid 2,3-dihydroxypropyl ester;
10) 2-hydroxy-4-[2-hydroxyimino-2-(5,5,8,8-tetramethyl-5,6,7,8-tetrahydronaphthalen-2-yl)ethoxy]benzoic acid 4-hydroxybutyl ester;
11) 2-hydroxy-4-[2-hydroxyimino-2-(5,5,8,8-tetramethyl-5,6,7,8-tetrahydronaphthalen-2-yl)ethoxy]benzoic acid 6-galactosyl ester;
12) 2-hydroxy-4-[2-hydroxyimino-2-(5,5,8,8-tetramethyl-5,6,7,8-tetrahydronaphthalen-2-yl)ethoxy]benzoic acid 6-glucosyl ester;
13) 2-hydroxy-4-[2-hydroxyimino-2-(5,5,8,8-tetramethyl-5,6,7,8-tetrahydronaphthalen-2-yl)ethoxy]benzoic acid 6-mannosyl ester;
14) 2-hydroxy-4-[2-hydroxyimino-2-(5,5,8,8-tetramethyl-5,6,7,8-tetrahydronaphthalen-2-yl)ethoxy]benzoic acid 2-amino-2-carboxyethyl ester;
15) 2-hydroxy-4-[2-hydroxyimino-2-(5,5,8,8-tetramethyl-5,6,7,8-tetrahydronaphthalen-2-yl)ethoxy]benzoic acid 4-(2-amino-2-carboxyethyl)phenyl ester; and
16) 2-hydroxy-4-[2-hydroxyimino-2-(5,5,8,8-tetramethyl-5,6,7,8-tetrahydronaphthalen-2-yl)ethoxy]benzoic acid 2-amino-2-carboxypropyl ester.

10. A cosmetic/pharmaceutical composition comprising at least one biaromatic compound of formula (I) as defined in claim 1, formulated into a cosmetically/pharmaceutically acceptable carrier therefor.

11. The cosmetic/pharmaceutical composition as defined in claim 10, formulated for topical application.

12. A regime or regimen for combating skin aging, comprising topically applying onto the skin of an individual in need of such treatment, a thus effective amount of the cosmetic/pharmaceutical composition as defined by claim 11.

13. A regime or regimen for treating a pathology for which a selective agonist activity with respect to the RAR-gamma receptor is desired, comprising administering to an individual in need of such treatment, a thus effective amount of at least one biaromatic compound of formula (I) as defined in claim 1.

14. The regime or regimen as defined by claim 13, comprising treating a pathology related to a cell differentiation disorder.

15. The regime or regimen as defined by claim 13, comprising treating a pathology related to a keratinization disorder.

16. The regime or regimen as defined by claim 13, comprising treating acne.

17. The regime or regimen as defined by claim 13, comprising treating psoriasis.

18. A process for preparing a compound of formula (I) as defined in claim 1, comprising conversion of the ketone function of the compound of formula 3: 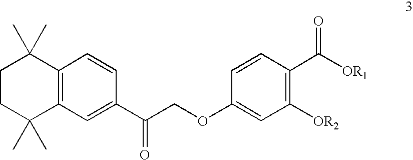
in which:
R₁ is an alkyl radical having from 2 to 20 carbon atoms, an alkenyl radical having from 2 to 20 carbon atoms, a mono- or polyhydroxyalkyl radical having from 1 to 6 carbon atoms, a sugar residue, or an amino acid residue; and
R₂ is a hydrogen atom or an alkyl radical having from 1 to 3 carbon atoms, to an oxime function, to produce the compound of formula (I):

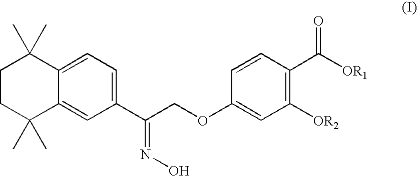

19. The process as defined by claim 18, wherein the oxime is prepared by reaction of the compound 3 with a mixture of hydroxylamine hydrohalide and of pyridine.

20. The process as defined by claim 19, wherein the compound 3 is prepared according to a process comprising alkylation of the compound 2: 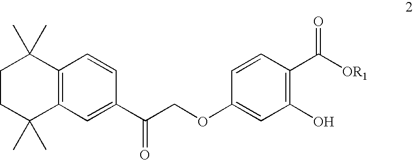
in the presence of a halide R₂X, wherein R₂ is an alkyl radical having from 1 to 3 carbon atoms, and X is a halogen atom.
